(19) United States
(12) Patent Application Publication
(10) Pub. No.: US 2022/0178911 A1
(43) Pub. Date: Jun. 9, 2022

(54) Synthetic Lethality Screening Platform for Cells Undergoing ALT

(71) Applicant: The Regents of the University of California, Oakland, CA (US)

(72) Inventors: Dirk Friedrich Hockemeyer, Berkeley, CA (US); Timothy K. Turkalo, Berkeley, CA (US)

(73) Assignee: The Regents of the University of California, Oakland, CA (US)

(21) Appl. No.: 17/681,714

(22) Filed: Feb. 26, 2022

Related U.S. Application Data
(63) Continuation of application No. PCT/US20/49616, filed on Sep. 7, 2020.
(60) Provisional application No. 62/898,502, filed on Sep. 10, 2019.

(51) Int. Cl. G01N 33/50; C12N 5/0735; C12N 15/10; C12N 15/11; C12N 15/90; C12N 9/22

(52) U.S. Cl. CPC G01N 33/5023; C12N 5/0606; G01N 33/5073; C12N 15/1093; C12N 2310/20; C12N 15/907; C12N 9/22; C12N 2510/04; C12N 2800/80; C12N 15/11

(57) ABSTRACT

An in vitro culture system of human embryonic stem (hES) derived cells is used as a synthetic lethality screening platform for cells undergoing alternative lengthening of telomeres (ALT).

This invention was made with government support under Grant Number CA196884 awarded by the National Institutes of Health. The government has certain rights in the invention.

### INTRODUCTION

Cancer cells can immortalize by inducing the recombination-based alternative lengthening of telomeres (ALT) pathway. Current methods of producing de novo cell lines which utilize the ALT mechanism of telomere elongation require first transforming a mortal cell line (e.g. IMR90) with SV40 large T antigen followed by passaging the culture through weeks-to-months of telomere crisis, after which individual cell clones will grow out of the culture. The immortalization efficiency of this process is low. Genetic inactivation by genome editing (knockout) of ATRX in these fibroblast cell lines will increase the rate at which the cells will activate ALT rather than reactivate telomerase expression, but even in this case the culture will still go through an extended crisis phase before individual clones activate ALT and resume proliferation.

### SUMMARY OF THE INVENTION

In an aspect the invention provides an in vitro culture of human embryonic stem derived cells immortalized through the alternative lengthening of telomeres (ALT) pathway, wherein the cells comprise:

(a) a genetic disruption of TERT locus that is:

(i) a heterozygous genetic disruption of the TERT locus; or

(ii) a homozygous genetic disruption of the TERT locus and a heterologous TERT allele operable as a conditional allele;

(b) a genetic disruption of the CDKN2A locus; and
(c) a genetic disruption of the ATRX locus,

wherein the culture is configured and operative as a synthetic lethality screening platform to identify gene products or drugs that interfere with ALT or homologous recombination.

In embodiments, the cells comprise:

(i) a heterozygous genetic disruption of the TERT locus, wherein: TERT was heterozygously knocked out by targeted excision of the TERT promoter using CAS9 and two guide RNAs surrounding the TERT promoter;

(ii) a homozygous genetic disruption of the TERT locus and a heterologous TERT allele operable as a conditional allele, wherein: a hygromycin selection cassette is integrated into the first exon of the TERT gene;

(ii) a homozygous genetic disruption of the TERT locus and a heterologous TERT allele operable as a conditional allele, wherein: a floxed TERT allele cassette is integrated at the AAVS1 safe harbor locus, operable as a conditional allele that can be excised to remove the TERT gene;

(b) a genetic disruption of the CDKN2A locus; and wherein the genetic disruption is of exon 2 the CDKN2A locus;

(c) a genetic disruption of the ATRX locus, wherein the genetic disruption is of exon 1 of the ATRX locus;

(d) a genetic disruption of the TP53 gene;

(d) a genetic disruption of the TP53 gene, wherein: a puromycin cassette is integrated into exon 4 of the TP53 gene;

an activated ALT pathway; and/or

an activated ALT pathway independent of TERT expression.

In an aspect the invention provides a method of using a subject culture comprising: (a) contacting the cells with an agent; and (b) detecting an effect of the agent on ALT activity in the cells.

In embodiments the method comprises: (i) screening for genetic interactors by genome-wide KO/siRNA libraries to determine novel targets; (ii) small molecule screening to identify novel chemotherapeutics for treating ALT cancers; or (iii) arrayed imaging screening to identify genetic interactors necessary for ALT activity;

In an aspect the invention provides a method for making a subject culture comprising:

(a) making a genetic disruption of TERT locus that is:

(i) making a heterozygous genetic disruption of the TERT locus; or

(ii) making a homozygous genetic disruption of the TERT locus and a heterologous TERT allele operable as a conditional allele;

(b) making a genetic disruption of the CDKN2A locus; and
(c) making a genetic disruption of the ATRX locus.

In embodiments the method comprises steps:

a) genetically disrupt the human TERT gene at the endogenous locus using zinc finger nuclease (ZFN) mediated integration of a hygromycin selection cassette into the first exon of TERT;
b) insert a floxed TERT allele at the AAVS1 safe harbor locus by ZFN-mediated integration, wherein this cassette functions as a conditional allele that can be excised to remove the TERT gene, and ensures that all cells immortalize through the ALT process;
c) genetically disrupt TP53 using ZFN mediated targeted integration of a puromycin cassette into the exon 4 of the TP53 gene;
d) genetically disrupt exon 2 of the CDKN2A locus by Cas9-mediated genome editing, wherein this locus encodes both p14 and p16 tumor suppressors which can be activated as a consequence of telomere shortening and telomere dysfunction; and
e) genetically disrupt ATRX by Cas9-mediated removal of the majority of the first coding exon of ATRX.

In embodiments the method further comprises: inducing ALT, comprising: removing the TERT transgene from the AAVS1 locus by expressing Cre recombinase and culturing of the resulting TERT-, TP53-, CDKN2A-, ATRX-stem cells in a stem cell media with reduced growth factors to induce differentiation.

The invention encompasses all combinations of the particular embodiments recited herein, as if each combination had been laboriously recited.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

We disclose a method to generate new ALT cell lines at a high rate that provides a platform for discovery. We demonstrate across multiple human ES cell lines the capability of activating the ALT process upon differentiation.

We have established and disclose a cell system in which ALT is induced with high frequency and without cells going through cellular crisis. The invention provides methods and compositions related to ALT-screening platforms, including novel methods of robustly generating ALT+ cells from genomically normal cells, and related ALT screening platforms. The ALT+ cells are early stage ALT, still exhibit significant genomic instability and exhibit features of ALT cancer cells and have wild-type isogenic control line that does not. Synthetic lethality/ALT inhibition by loss of other gene product can be assayed for by multiple methodologies.

We also show that excising the floxed TERT allele in the AAVS1 is not necessary to activate ALT and that ALT activity is fully compatible with TERT expression.

In embodiments we demonstrate that TERT expression has no bearing on the ability of the cell to activate the ALT pathway; hence our claims encompass ALT utilization in the heterozygous TERT line, the conditional TERT line with TERT removed, and in the conditional TERT line with TERT still remaining.

Our system can act as a platform not only for synthetic lethality for ALT screening, but as a platform to screen for novel recombination factors and chemical interventions that interfere with recombination. Chemical intervention in the recombination pathway is way to induce synthetic lethality in DNA repair deficient cancer cells.

Applications of the invention include identification of cancer therapeutics by means of: Screening for genetic interactors by genome-wide KO/siRNA libraries to determine novel targets; small molecule screening to identify novel chemotherapeutics for treating ALT cancers; and arrayed imaging screening to identify genetic interactors necessary for ALT activity.

In embodiments our cells are genomically normal until the point that they activate the ALT mechanism, and due to the population-wide nature of the immortalization process, they do not carry the genomic idiosyncrasies of a clonally derived cancer cell line. Because they can immortalize and exhibit features of ALT by a variety of differentiation paradigms in tissues of multiple germ layers, the features they exhibit are those general to ALT and not specific to the cell line. Therefore, they provide a platform for screening to produce targets for ALT therapeutics.

One such screening method is genome wide CAS9 screens. Since the development of CAS9 as a genome editing tool and its simplicity of targeting, it has been possible to generate genome wide libraries for CRISPR knockout as well as those for knockdown and overexpression. In brief, these tools allow for near-comprehensive analysis of genetic interactions by either knocking out, reducing expression of, or increasing expression of every gene within a large population of cells.

Another screen is for synthetic lethality with ALT, in which a CRISPR knockout library is delivered to a population of recently immortalized ALT cells and isogenic wildtype controls. Timepoints can be taken immediately after transduction to determine the initial complexity, and then days-to-weeks later to allow for time for synthetic lethality to occur. Guides which deplete within ALT cells but not wildtype cells are potential targets for therapeutics, as it indicates that those are genes which ALT cells specifically require for continued proliferation. Likewise, complementary CRISPRa and CRISPRi libraries can be utilized to discover other pathways which interact with alternative telomere lengthening and proliferation of ALT cells.

Furthermore, applications are not limited to assaying for cell viability in genome wide screens. In addition to screening using depletion and enrichment of guides determined by sequencing, it is also an option to perform genome wide imaging-based screening within the ALT cells our immortalization method produces (Wang et al., 2019, PNAS USA 116, 10842-10851). ALT cells have several phenotypes assayable by simple imaging readout. For instance, ALT cells exhibit colocalization of PML protein with telomeres (ALT-associated PML bodies, or APBs) as well as novel nucleotide incorporation at telomeres outside of S-phase, visualizable through treatment with EdU. We examined these phenotypes: they are simple, readily automatable staining assays, and the arrayed imaging is readily scaled to a genome wide knockout library to investigate the interactions between genes that can lead to the abrogation of these ALT phenotypes.

Additionally, this imaging-based screening provides more meaningful information than a cell viability screen. While a standard synthetic lethal screen will only inform us of the genes necessary for proliferation, the imaging-based screen provides for the identification of factors necessary for the formation of ALT-specific phenotypes; for example, targeting a factor necessary for the agglomeration of telomeres into an APB may be less toxic to normal cells than a factor necessary for general cell proliferation.

### Example

We genetically engineered human embryonic stem cells (e.g. WIBR #3, NIH stem cell registry 0079), followed by growth factor withdrawal induced differentiation. The WIBR #3 cells carry the following genetic modifications:

Genetic disruption of the human TERT gene at the endogenous locus using zinc finger nuclease (ZFN) mediated integration of a hygromycin selection cassette into the first exon of TERT.

Expression of a floxed TERT allele at the AAVS1 safe harbor locus, inserted by ZFN-mediated integration. This cassette functions as a conditional allele that can be excised to remove the TERT gene. This will ensure that all cells immortalize through the ALT process.

Genetic disruption of TP53 using ZFN mediated targeted integration of a puromycin cassette into the exon 4 of the TP53 gene. Note: TP53 is mutated in the majority of ALT cancers and this genetic alteration recapitulates this state.

Genetic deletion of exon 2 of the CDKN2A locus by Cas9-mediated genome editing. This locus encodes both p14 and p16 tumor suppressors which can be activated as a consequence of telomere shortening and telomere dysfunction.

Genetic disruption of ATRX by Cas9-mediated removal of the majority of the first coding exon of ATRX.

To induce ALT, we removed the TERT transgene from the AAVS1 locus by expressing Cre recombinase and culture of the resulting TERT-, TP53-, CDKN2A-, ATRX-stem cells in a stem cell media with reduced growth factors to induce differentiation. These differentiated cells acquire ALT characteristics (e.g. long, heterogeneous telomeres, ALT-associated colocalizations of PML protein and telomeres [APBs], circular extrachromosomal telomeric repeats [C-circles]) and exhibit continuous proliferation long after the matched hESC cultures go through telomere shortening-induced crisis.

Our method causes cell cultures to experience about a week of slower proliferation, but recovery of the majority of cells which activate ALT and contribute to the culture's growth. Generally more that 25% of the cells effectively induce ALT and have not undergone cellular crisis. Thus, the ALT cells produced are polyclonal rather than monoclonal.

Cell Culture

Genome editing was performed in WIBR 3 hESCs, NIH stem cell registry #0079 (Lengner et al., 2010, Cell 141, 872-883). Cell culture was carried out as previously described (Soldner et al., 2009, Cell 136, 964-977). Briefly, hESC lines were maintained on a monolayer of CD-1 strain mouse embryonic fibroblasts (MEFs) [Charles River] inactivated by 35 Gy of γ-irradiation. hESCs were grown in hESC medium (DMEM/F12 [Gibco] supplemented with 20% KnockOut Serum Replacement [Gibco], 1 mM glutamine [Sigma-Aldrich], 1% non-essential amino acids [Gibco], 0.1 mM β-mercaptoethanol [Sigma-Aldrich], 100 U/mL Penicillin-Streptomycin [Gibco], and 4 ng/mL FGF-Basic (AA 1-155) [Gibco]). Cultures were passaged every 5-7 days either manually or enzymatically with 1.5 mg/mL collagenase type IV [Gibco] by sedimentation and washing 3 times in wash medium (DMEM [Gibco] supplemented with 5% newborn calf serum [Sigma-Aldrich] and 100 U/mL Penicillin-Streptomycin [Gibco]).

HeLa 1.3 cervical carcinoma cells (The Rockefeller University, New York, N.Y.) (Takai et al., 2009, JBC 285, 1457-1467). U2OS osteosarcoma cells were obtained from the UC Berkeley Cell Culture Facility. HeLa 1.3 and U2OS were maintained in fibroblast medium (DMEM [Gibco] supplemented with 15% FB Essence [Seradigm], 1 mM glutamine [Sigma-Aldrich], 1% non-essential amino acids [Gibco], and 100 U/mL Penicillin-Streptomycin [Gibco]) and passaged every 3-5 days enzymatically with Trypsin-EDTA (0.25%) [Gibco]. Trypsin was inactivated by either wash medium or fibroblast medium.

Transient feeder-free hESC and E7-differentiated cultures were maintained in “E7” medium (DMEM/F12 [Gibco] supplemented with 10% FB Essence [Seradigm], 64 mg/L L-ascorbic acid [Sigma-Aldrich], 14 μg/L sodium selenium [Sigma-Aldrich], 100 μg/L FGF-Basic (AA 1-155) [Gibco], 19.4 mg/L insulin [Sigma-Aldrich], 543 mg/L NaHCO₃ [Sigma-Aldrich], and 10.7 mg/L transferrin [Sigma-Aldrich]. Cells were grown on tissue culture plates treated with Matrigel matrix [Corning].

Genome Editing in hESCs

All targeting was performed as previously described (Hockemeyer et al., 2009 Nat Biotechnol 27, 851-857; Hockemeyer et al., 2011 Nat Biotechnol 29, 731-734; Chiba and Hockemeyer, 2014). ZFNs targeting TERT and TP53 were obtained from Sangamo Therapeutics, Inc. ZFN and TALEN targeting was performed as previously described. CAS9 and sgRNAs were expressed using the pX330 plasmid (Le Cong et al., 2013 Science (New York, N.Y.) 339, 819-823). Each targeting step was performed by co-electroporation of 1-2×10⁷ hESCs with 15 μg of each pX330 plasmid and 7.5 μg of GFP expression plasmid. 48-72 hours later cells were sorted for GFP fluorescence and single cell-derived hESC colonies were isolated and genotyped by Southern blotting or PCR followed by Sanger sequencing.

Cre-mediated and Flp-mediated recombination was performed by co-transfection of StemMACS Cre recombinase mRNA [Milltenyi Biotec] or StemMACS Flp recombinase mRNA [Milltenyi Biotec] with Stemgent eGFP mRNA [Milltenyi Biotec] into hESCs using StemFect RNA Transfection Kit [ReproCELL] according to manufacturer instructions. 24-72 hours later cells were sorted for GFP fluorescence and single cell-derived hESC colonies were isolated and genotyped by PCR.

Southern Blotting and PCR Genotyping

Southern blot analysis was performed as previously described (Hockemeyer et al., 2009 Nat Biotechnol 27, 851-857; Hockemeyer et al., 2011 Nat Biotechnol 29, 731-734; Sexton et al., 2014 Genes Dev 28, 1885-1899). TP53 deletion was confirmed using an external 5′ probe amplified from genomic DNA with primers. CDKN2A deletion was confirmed using a probe 5′ to the excision site amplified from genomic DNA with primers. ATRX deletion was confirmed using PCR primers. ATRX conditional reintroduction was confirmed using PCR primers. Cre-mediated loopout of TERT from AAVS1 was confirmed using PCR primers.

Fibroblast Differentiation

hESC colonies were lifted from the MEF feeder layer enzymatically with 1.5 mg/mL collagenase type IV [Gibco] and isolated by sedimentation and washing 3 times with wash medium. Colonies were suspended in fibroblast medium and grown in Ultra-Low Attachment Culture Dishes [Corning] for formation of embryoid bodies (EBs). Medium was replenished every 3 days by sedimentation and resuspension of EBs. After 9 days EBs were transferred to tissue culture dishes to attach. 7 days later, EBs and fibroblast-like cells were passaged using Trypsin-EDTA (0.25%) [Gibco], triturated to single-cell suspension, and plated on tissue culture dishes. Cultures were maintained in fibroblast medium on plates treated with gelatin [Sigma-Aldrich]and were passaged every 5-7 days.

E7 Differentiation

hESC colonies were lifted from the MEF feeder layer enzymatically with 1.5 mg/mL collagenase type IV [Gibco] and isolated by sedimentation and washing 3 times with wash medium. Colonies were suspended in E7 medium and transferred to tissue culture dishes treated with Matrigel [Corning]. After 7 days, cultures were passaged using Trypsin-EDTA (0.25%) [Gibco], triturated to single-cell suspension, and plated on Matrigel-coated tissue culture dishes. Cultures were maintained in E7 medium and were passaged every 5-7 days.

Neural Precursor Cell Differentiation

Single-cell dissociated hESCs were cultured on Matrigel-coated plates at a density of 5×10⁴ cells/cm² and maintained in complete conditioned hESC medium until >90% confluent. A modified dual-SMAD inhibition protocol was performed to differentiate hESCs into NPCs as described previously (Blair et al., 2018 Nat Med 24, 1568-1578. Cells were passaged by dissociation with StemPro Accutase, split 1:3 every 5 days, and maintained in N2 medium (50% DMEM/F12 [Gibco] and 50% Neurobasal Medium [Gibco] supplemented with N-2 Supplement [Gibco], GlutaMAX [Gibco], 100 U/mL Penicillin-Streptomycin [Gibco], 0.2% insulin [Sigma-Aldrich], and 0.075% (w/v) bovine serum albumin [Sigma-Aldrich].

Teratoma Formation

hESCs were collected by collagenase type IV (1.5 mg/mL) treatment and separated from MEF feeder cells by sedimentation. Cells were resuspended in 250 μL of hESC medium and injected subcutaneously into NOD-SCID mice [Taconic Biosciences]. Tumors which grew to the maximum size of 2.5 cm were explanted, measured, and divided for frozen sections and formalin fixation.

Immunocytochemistry (ICC) and EdU Staining

Cells were plated on to glass coverslips and fixed in 4% paraformaldehyde solution in PBS [Sigma-Aldrich]. Samples were permeabilized in permeabilization solution (PBS with 3% horse serum [Sigma-Aldrich] and 0.1% Triton X-100 [Sigma-Aldrich]). Primary antibody incubation was performed overnight in permeabilization solution at 4° C. Samples were then washed 3 times in PBS, 5 minutes each wash. Secondary antibody incubation was performed for 1 hour in permeabilization solution at 25° C. protected from light. Samples were washed 3 times in PBS, 5 minutes each wash, with 1 μg/mL DAPI [KPL] added to the second wash. Coverslips were then mounted in ProLong Gold Antifade mountant on glass slides and imaged.

For EdU staining, cells were plated on glass coverslips. 24 hours prior to fixation, cells were treated with 10 μM RO-3306 [Sigma-Aldrich]. 2 hours prior to fixation, 10 μM EdU was added to cell culture media. Samples were then fixed in 4% paraformaldehyde solution in PBS. EdU detection was then performed using the Click-iT Plus EdU Alexa Fluor 647 Imaging Kit [Thermo Fisher]. For further ICC steps, samples were subsequently treated as described above.

C-Circle Assay.

The C-circle assay was performed as previously described (Henson et al., 2009 Nat Biotechnol 27, 1181-1185). Briefly, extracted genomic DNA was digested in EcoRI [New England BioLabs], precipitated and extracted by phenol-chloroform, resuspended, and quantified by a Qubit™ 2.0 Fluorometer [Life Technologies]. 20 ng of each sample was incubated with φ29 polymerase as previously described. Samples were attached to an Amersham Hybond-XL membrane [Fisher Scientific] by dot blot and probed with a ³²P-end-labeled oligonucleotide. Parallel membranes were probed with a 5′-gtaatcccagcactttgg-3′ end-labeled oligonucleotide which binds to the Alu consensus sequence to normalize for genomic DNA content.

Chromosome-Orientation Fluorescence In Situ Hybridization (CO-FISH)

CO-FISH analysis was performed as previously described (Williams and Bailey, 2010 Cold Spring Harb Protoc 2009, pdb.prot5269). Briefly, 24 hours prior to fixation, cells were cultured in growth medium containing 10 μM bromodeoxyuridine (BrdU) [Invitrogen]. 2 hours prior to fixation, 0.2 μg/mL colcemid [Roche] was added to medium. Cells were dissociated by Trypsin-EDTA (0.25%) [Gibco], centrifuged and gently resuspended for 5 minutes in 75 mM KCl. Cells were then centrifuged, supernatant was aspirated, and cells were gently resuspended in residual supernatant before fixation in 3:1 methanol:acetic acid. Metaphase spreads were made according to standard techniques and BrdU-containing strands were digested according to the Williams and Bailey 2010 protocol cited above. Telomeres were hybridized sequentially with TelG-Alexa488 and TelC-Cy3 (PNA Bio). After dehydration, slides were stained with 1 μg/mL DAPI [KPL] and mounted with ProLong Gold mountant before imaging.

Image Acquisition and Analysis

All ICC and CO-FISH micrographs were taken on a Nikon Eclipse TE2000-E with a 100× objective using an Andor Zyla 4.2 sCMOS camera. Images were acquired using Nikon NIS-Elements. For experiments in which two or more conditions were quantitatively compared the same exposure and acquisition settings were used for each image. APB, TIF, and EdU colocalization with telomeres were automatically processed using ImageJ with ComDet plugin.

Strand-Seq, Library Preparation, Illumina Sequencing, and Bioinformatics

Cells were prepared for Strand-seq analysis as previously described (Sanders et al., 2017 Nat Protoc 12, 1151-1176). Briefly, hESCs were collected after BrdU pulse and resuspended in nuclei staining buffer (100 mM Tris-HCl pH 7.4, 150 mM NaCl, 1 mM CaCl₂, 0.5 mM MgCl₂, 0.1% NP-40, and 2% bovine serum albumin [Sigma-Aldrich] supplemented with 10 μg/mL Hoechst 33258 [Enzo] and 10 μg/mL propidium iodide [Sigma-Aldrich]. Single nuclei were sorted into 5 μL ProFreeze-CDM freeze medium [Lonza]+7.5% dimethyl sulfoxide in 96-well skirted PCR plates [4titude], based on low propidium iodide and low Hoechst fluorescence using an Influx cell sorter [BD Biosciences]. For each experiment, 96 libraries were pooled and 250-450 bp-sized fragments were isolated and purified. DNA was processed for Strand-seq (Sanders et al., 2017 Nat Protoc 12, 1151-1176). DNA quality and concentrations were assessed using the High Sensitivity dsDNA kit [Agilent] on the Agilent 2100 Bio-Analyzer and on a Qubit 2.0 Fluorometer [Life Technologies].

Clusters were generated on the cBot (HiSeq2500) and single-end 50 bp reads were generated using the HiSeq2500 sequencing platform [Illumina]. Indexed bam files were aligned to human (GRCh37) using Bowtie2 (Langmead and Salzberg, 2012). SCEs were identified and mapped with the BAIT software package using standard settings.

Telomere Length Assessment

To collect genomic DNA, hESC lines were enzymatically released from the MEF feeder layer by treatment with 1.5 mg/mL collagenase type IV and washed 3 times in wash medium and gravitational sedimentation to minimize contaminating MEF cells. Genomic DNA was prepared as described previously (Hockemeyer et al., 2005 EMBO J 24, 2667-2678). MEF telomeres are resolved by size from hESC telomeres and do not interfere with analysis of telomere length. Genomic DNA was digested with MboI and AluI overnight at 37° C. Digested DNA was normalized and run on a 0.75% Seakem ME Agarose [Lonza] gel and dried under vacuum for 2 hours at 50° C. The dry gel was denatured in 0.5 M NaOH, 1.5 M NaCl for 30 minutes at 25° C., then neutralized with 1 M Tris-HCl pH 6.0, 2.5 M NaCl, 2× for 15 minutes. The gel was then pre-hybridized in Church's buffer (1% BSA, 1 mM EDTA, 0.5 M NaPO₄, 7% SDS, pH 7.2) for 1 hour at 55° C. before adding ³²P-end-labeled (CCCTAA)₃ probe. The gel was washed in 4×SSC buffer 3 times for 15 minutes at 50° C. and once in 4×SSC+0.1% SDS at 25° C. before exposing on a phosphorimager screen.

Single telomere length analysis (STELA) was performed as previously described (Baird et al., 2003 Nat Genet 33, 203-207). hESC colonies were separated from the MEF layer by treatment with 1.5 mg/mL collagenase type IV and washed 3× in wash medium, collecting by sedimentation to minimize contaminating MEF cells. DNA was extracted from cell pellets using the Norgen Cells and Tissue DNA Isolation Micro Kit. DNA was solubilized by digestion with EcoRI and quantified on a Qubit 2.0 Fluorometer, then diluted to 10 ng/μL in 10 mM Tris-HCl (pH 7.5). DNA was ligated at 35° C. for 12 hours in a volume of 10 μL containing 10 ng genomic DNA, 0.9 μM telorette linker, and 0.5 U T4 DNA ligase [New England Biolabs]. Ligated DNA was diluted to 250 pg/μL in water and multiple PCRs were performed in volumes of 15 μL containing 200 pg ligated DNA, 0.25 μM XpYpE2+G and teltail primers, 0.3 mM dNTPs, 7.4 mM MgCl₂, 1×Taq Buffer with (NH₄)₂SO₄, and 1 U of a 10:1 mix of Taq [New England Biolabs] and Pwo [Sigma-Aldrich] polymerase. Reactions were cycled on a Bio-Rad C1000 Touch Thermal Cycler: 25 cycles of 94° C. for 15 seconds, 65° for 30 seconds, 68° for 10 minutes. DNA fragments were resolved on a 0.5% agarose gel and detected by Southern blot with a random-primed α-³²P-labeled XpYp probe generated by PCR using primers (XpYpE2; XpYpB2). Telomere lengths were estimated using TeSLA-QUANT software (Lai et al., 2017 Nat Commun 8, 1356).

Immunoblotting

Cells were collected RIPA buffer (150 mM NaCl, 1% Triton X-100, 0.5% sodium deoxycholate, 0.1% SDS, 50 mM Tris pH 8.0) with 1 mM phenylmethanesulfonyl fluoride and cOmplete ULTRA protease inhibitor [Roche] and Halt Phosphatase inhibitor [Thermo Scientific]. Protein concentration was determined by Bio-Rad Protein Assay colorimetric dye quantified by a Bio-Rad xMark microplate reader. 15-20 μg protein in Laemmli sample buffer was loaded onto 5% (ATRX) or 10% (DDR proteins) polyacrylamide gels. Proteins were transferred to nitrocellulose membranes [Bio-Rad], blocked in 5% BSA in tris-buffered saline (TBS)-Tween 20 for 1 hour at 25° C., then incubated with primary antibodies diluted in 5% BSA in TBS-T overnight at 4° C. Membranes were then washed 3×15 minutes in TBS-T and incubated in horseradish peroxidase-conjugated secondary antibodies [Bio-Rad] for 1 hour at 25° C., washed, incubated with Clarity Western ECL substrate [Bio-Rad] before imaging on a Bio-Rad ChemiDoc XRS+. Membranes were stripped by 2×10 minute incubation at 25° C. in stripping buffer (200 mM glycine, 0.1% SDS, 1% Tween 20, pH 2.2) before re-blocking and incubation with subsequent primary antibodies.

qRT-PCR Analysis

RNA was isolated using Trizol [Invitrogen] extraction followed by ethanol precipitation. Reverse transcription was performed on 1000 ng of total RNA by oligo(dT) and random priming using the iScript cDNA Synthesis Kit [Bio-Rad]. qRT-PCR was performed in a CFX96 [Bio-Rad] with KAPA SYBR FAST master mix ROX low [Roche].

Telomerase Catalytic Activity Assay

PCR-based telomeric repeat amplification protocol (TRAP) was performed as previously described (Xin, 2011 Methods Mol Biol 735, 107-111). Protein extracts were generated by repeated freeze-thaw cycles in hypotonic lysis buffer (HLB) (20 mM HEPES, 2 mM MgCl₂, 0.2 mM EGTA, 10% glycerol, 1 mM dithiothreitol, 0.1 mM PMSF, 0.5% CHAPS). Protein concentrations were determined by Bio-Rad Protein Assay colorimetric dye quantified by a Bio-Rad xMark microplate reader. 200 ng of total protein were used for input into ³²P-dGTP PCR. TRAP products were resolved on a 10% polyacrylamide in 1×TAE gel. Dried gels were visualized by exposure on a phosphorimager screen.

hESC Colony Cell Counting

To measure hESC population doubling, hESC colonies were grown feeder-free in E7 medium supplemented with ROCK inhibitor (Y-27632) [Chemdea] for 24 hours, then treated with Trypsin-EDTA (0.25%) for single-cell suspension. hESCs were plated at low density (1000 cells/10 cm plate) on tissue culture plates coated with Matrigel. After 72 hours, cells were washed with PBS and fixed with 4% paraformaldehyde in PBS. Nuclei were counterstained with 1 μg/mL DAPI. Distinct clonal colonies were imaged and nuclei counted. Population doublings were calculated assuming colonies were founded by single cells.

1. An in vitro culture of human embryonic stem derived cells immortalized through the alternative lengthening of telomeres (ALT) pathway, wherein the cells comprise:
(a) a genetic disruption of TERT locus that is:
(i) a heterozygous genetic disruption of the TERT locus; or
(ii) a homozygous genetic disruption of the TERT locus and a heterologous TERT allele operable as a conditional allele;
(b) a genetic disruption of the CDKN2A locus; and
(c) a genetic disruption of the ATRX locus,
wherein the culture is configured and operative as a synthetic lethality screening platform to identify gene products or drugs that interfere with ALT or recombination.

2. The in vitro culture of claim 1, wherein the cells comprise:
(i) a heterozygous genetic disruption of the TERT locus, wherein:
TERT was heterozygously knocked out by targeted excision of the TERT promoter using CAS9 and two guide RNAs surrounding the TERT promoter.

3. The in vitro culture of claim 1, wherein the cells comprise:
(ii) a homozygous genetic disruption of the TERT locus and a heterologous TERT allele operable as a conditional allele, wherein:
a hygromycin selection cassette is integrated into the first exon of the TERT gene.

4. The in vitro culture of claim 1, wherein the cells comprise:
(ii) a homozygous genetic disruption of the TERT locus and a heterologous TERT allele operable as a conditional allele, wherein:
a floxed TERT allele cassette is integrated at the AAVS1 safe harbor locus, operable as a conditional allele that can be excised to remove the TERT gene;

5. The in vitro culture of claim 1, wherein the cells comprise:
(b) a genetic disruption of the CDKN2A locus; and
wherein the genetic disruption is of exon 2 the CDKN2A locus.

6. The in vitro culture of claim 1, wherein the cells comprise:
(c) a genetic disruption of the ATRX locus,
wherein the genetic disruption is of exon 1 of the ATRX locus.

7. The in vitro culture of claim 1, wherein the cells comprise:
(d) a genetic disruption of the TP53 gene.

8. The in vitro culture of claim 1, wherein the cells comprise:
(d) a genetic disruption of the TP53 gene, wherein:
a puromycin cassette is integrated into exon 4 of the TP53 gene.

9. The in vitro culture of claim 1, wherein the cells comprise an activated ALT pathway.

10. The in vitro culture of claim 1, wherein the cells comprise an activated ALT pathway independent of TERT expression.

11. A method of using the culture of claim 1, comprising:
(a) contacting the cells with an agent; and
(b) detecting an effect of the agent on ALT activity in the cells.

12. The method of claim 11, comprising:
(a) contacting the cells with an agent; and
(b) detecting an effect of the agent on ALT activity in the cells; wherein the method comprises:
(i) screening for genetic interactors by genome-wide KO/siRNA libraries to determine novel targets;
(ii) small molecule screening to identify novel chemotherapeutics for treating ALT cancers; or
(iii) arrayed imaging screening to identify genetic interactors necessary for ALT activity.

13. A method for making the culture of claim 1, comprising:
(a) making a genetic disruption of TERT locus that is:
(i) a heterozygous genetic disruption of the TERT locus; or
(ii) a homozygous genetic disruption of the TERT locus and a heterologous TERT allele operable as a conditional allele;
(b) making a genetic disruption of the CDKN2A locus; and
(c) making a genetic disruption of the ATRX locus.

14. The method of claim 13, comprising:
a) genetically disrupt the human TERT gene at the endogenous locus using zinc finger nuclease (ZFN) mediated integration of a hygromycin selection cassette into the first exon of TERT;
b) insert a floxed TERT allele at the AAVS1 safe harbor locus by ZFN-mediated integration, wherein this cassette functions as a conditional allele that can be excised to remove the TERT gene, and ensures that all cells immortalize through the ALT process;
c) genetically disrupt TP53 using ZFN mediated targeted integration of a puromycin cassette into the exon 4 of the TP53 gene;
d) genetically disrupt exon 2 of the CDKN2A locus by Cas9-mediated genome editing, wherein this locus encodes both p14 and p16 tumor suppressors which can be activated as a consequence of telomere shortening and telomere dysfunction; and
e) genetically disrupt ATRX by Cas9-mediated removal of the majority of the first coding exon of ATRX.

15. The method of claim 14 further comprising: inducing ALT, comprising: removing the TERT transgene from the AAVS1 locus by expressing Cre recombinase and culturing of the resulting TERT-, TP53-, CDKN2A-, ATRX-stem cells in a stem cell media with reduced growth factors to induce differentiation.
